(19) United States
(12) Patent Application Publication
(10) Pub. No.: US 2010/0159590 A1
(43) Pub. Date: Jun. 24, 2010

(54) SYSTEMS AND METHODS FOR ACTIVE MICROFLUIDIC CELL HANDLING

(75) Inventors: Ray Alley, Honolulu, HI (US); Maile Giffin, Kailua, HI (US); Len Higashi, Pearl City, HI (US); Luc Bousse, Los Altos, CA (US); Roger Rudoff, Cupertino, HI (US)

Correspondence Address: Convergent Law Group LLP, P.O. BOX 1329, MOUNTAIN VIEW, CA, 94042, US

(73) Assignee: NANOPOINT, INC., Honolulu, HI (US)

(21) Appl. No.: 12/443,148

(22) PCT Filed: Oct. 4, 2007
(86) PCT No.: PCT/US07/80483

§ 371 Date: Nov. 5, 2009

Related U.S. Application Data
(60) Provisional application No. 60/849,912, filed on Oct. 5, 2006.

(51) Int. Cl. C12N 5/02; C12M 1/22

(52) U.S. Cl. 435/374; 435/305.2

(57) ABSTRACT

A method and system for providing a cell support system are described. The method and system include providing a first block, a second block coupled to the first block, and a plurality of microfluidic channels. The first block includes a plurality of cell wells therein. The second block includes a plurality of through holes therethrough. The plurality of holes are in fluid communication with at least one corresponding cell well of the plurality of cell wells. The plurality of microfluidic channels are in fluid combination with at least a portion of the plurality of cell wells and are configured to provide an active fluid flow with the portion of the plurality of cell wells.

### BACKGROUND OF THE INVENTION

Growing and maintaining collections of cells in controlled environments are prerequisite steps for a variety of experimental procedures. Collections of cells may be studied in themselves or may be subjected to specific interventions whose consequences may then be evaluated. Collections of cells may be studied visually or optically, for example by counting the number or density of cells or their degree of confluence. Collections of cells may also be studied using other methods, for example by attaching fluorescent dyes or tags, by measuring metabolic activity, or by making electrochemical measurements. In addition, collections of cells may be grown for experimental or therapeutic purposes, for example in tissue engineering. A number of testing systems may take advantage of collections of cells in controlled environments, including monoclonal antibody selection, drug screening, protein-protein interaction assays, virus cell interactions, genetic tests, antibacterial assays, cytological tests, toxicological assays, and the like.

Biological research laboratories recognize the usefulness of systems and methods that make handling cell collections more efficient. A variety of screening tests, such as so-called “lab-on-a chip” technologies, depend upon rapid acquisition of biological information from cell collections. While systems and methods exist presently for these purposes, these technologies have limitations.

Typical containment devices for culturing and maintaining live cell collections for analysis include petri dishes, well plates and bath chambers. Many of these systems suffer from inconsistent arrangement of cells and slow processing times. Moreover, the arrangement of cells in such cell analysis systems may be random, with areas of high cell congregation and other regions where cells are sparsely distributed.

Well microtiter plates offer improved efficiencies as compared to more conventional techniques for processing live cells. Well microtiter plate technologies may be used to run assays for a variety of cellular studies. Typically, well microtiter plates come in sizes of 96, 384 and 1536 wells with a plate physical dimension of 128×86 mm. In these systems, each well must be filled with a certain number of live cells, so that the use of these well plates requires a significant number of cells and a significant amount of culture medium to sustain them. In addition, the feeding of the cells and removal of waste products is not easily performed on the microwell plate. Furthermore, if the cells on a well plate are to be used for a diagnostic test or to test a new drug, an appropriate volume of test reagents is required. Despite the improvements that these systems offer, the cost associated with running tests using this technology may be substantial. Moreover, tests using this technology may produce significant amounts of biohazard waste material.

To address some of these problems, microfluidic devices have been developed to carry out high-throughput testing at lower cost, particularly taking advantage of miniaturized form factor for cell analysis. As an example, microfluidic structures may provide parallel wells or basins for holding cells, or may provide narrow channels through which cells may circulate, permitting, for example, observing them with fluorescence, or performing electrical lysis on them to determine intracellular processes through chemical analysis.

As an example, McClain et. al. (M. A. McClain, C. T. Culbertson, S. C. Jacobson, N. L. Allbritton, C. E. Sims, J. M. Ramsey, “Microfluidic devices for the high throughput chemical analysis of cells” Analytical Chemistry (2003) 75: 5646-5655) developed a microfluidic device for high-throughput chemical analysis of cells. The device integrated cell handling, rapid cell lysis and electrophoretic separation and detection of fluorescent cytosolic dyes. However, this device was adapted for processes that involve lysing live cells, and it was not intended for live cell studies over extended periods.

As another example, U.S. Pat. No. 7,190,449 discloses a microarray that provides a two-dimensional array of cells in precise, equally spaced rectangular cubicles or cylindrical silos (otherwise referred to individual cell wells) that may contain culture medium. This microarray may be suitable for simultaneous monitoring and analyzing of a large matrix of cells, biological fluids, chemicals and/or solid samples.

Other microfluidic systems have been adapted for applications that include patterning or controlling the adhesion of cells on surfaces, moving cells through narrow channels (e.g., microfabricated flow cytometry), sustaining cells in polydimethylacrylamide (PDMA) structures, enclosing cells in larger, multi-well chambers, and growing cells under conditions that mimic those found in natural tissues or organs (i.e., tissue engineering).

There remains a need in the art for systems and methods that permit the economical and efficient growth and maintenance of cell collections.

### BRIEF SUMMARY OF THE INVENTION

Disclosed herein are embodiments of a cell support system that may include a first block including a plurality of cell wells therein; a second block coupled with the first block, the second block including a plurality of through holes therethrough, the plurality of through holes being in fluid communication with at least one corresponding cell well of the plurality of cell wells; and a plurality of microfluidic channels in fluid communication with at least a portion of the plurality of cell wells, the plurality of microfluidic channels configured to provide an active fluid flow with the portion of the plurality of cell wells. In embodiments, the plurality of microfluidic channels provides at least one of the active fluid flow to the portion of the plurality of cell wells and the active flow from the portion of the plurality of cell wells. In embodiments, the cell support system includes at least one manifold coupled with the plurality of microfluidic channels. In embodiments, the at least one manifold provides fluid communication with at least one external reservoir. In embodiments, the at least one access port provides access to the at least one manifold without interrupting its fluid communication with the plurality of microfluidic channels. The at least one manifold may isolate a first portion of the plurality of cell wells from a second portion of the plurality of cell wells. In embodiments, the manifold may interface with a fluid delivery system in fluid communication with the plurality of microfluidic channels and provides inflow fluid thereto.

The first block of the cell support system may include a first plurality of recesses and the second block includes a second plurality of recess substantially aligned with the first plurality of recesses to form the plurality of microfluidic channels. At least a portion of the plurality of microfluidic channels may be formed within the first block, or within the second block.

The plurality of microfluidic channels may include a plurality of inlet channels characterized by an inlet size and at least one main channel characterized by a main channel size, the at least one main channel size being at least an order of magnitude larger than the inlet channel size. In embodiments, the main channel size is at least two orders of magnitude larger than the inlet channel size.

In embodiments, the plurality of microfluidic channels may include at least one inlet, the plurality of microfluidic channels being configured to support a pressure differential between the at least one inlet and the portion of the plurality of cell wells.

In embodiments, the plurality of microfluidic channels are substantially horizontal and the plurality of cell wells is substantially vertical. In embodiments, the plurality of microfluidic channels are configured to be microfluidically closed and the plurality of cell wells are configured to be mechanically open. The plurality of microfluidic channels includes a plurality of inlet channels and at least one main channel at an angle with the plurality of inlet channels. In embodiments, this angle may be approximately ninety degrees.

Disclosed herein are embodiments of a cell support system including a first block including a plurality of cell wells and a first plurality of recesses therein; a second block, the second block including a plurality of through holes therein and a second plurality of recesses therein, the plurality of through holes being in fluid communication with at least one corresponding cell well of the plurality of cell wells, the second block being coupled with the first block such that the first plurality of recesses is substantially aligned with the second plurality of recesses to form a plurality of microfluidic channels having at least one inlet, the first block being coupled with the second block such that the plurality of cell wells are substantially aligned, the plurality of microfluidic channels configured to provide fluid communication with the portion of the plurality of cell wells, to provide a pressure differential between the at least one inlet and the portion of the plurality of cell wells, and to provide an active fluid flow with the portion of the plurality of cell wells.

Disclosed herein are embodiments of a cell support system including a first block including a plurality of cell wells; a second block including a plurality of through holes therein, the plurality of through holes being in fluid communication with at least one corresponding cell wells; and a plurality of microfluidic channels in fluid communication with at least a portion of the plurality of cell wells, the plurality of microfluidic channels having at least one inlet permitting fluid inflow and at least one outlet permitting fluid outflow, the plurality of microfluidic channels configured to provide a pressure differential between the at least one inlet and the portion of the plurality of cell wells, wherein the through holes permit mechanical access to the plurality of cell wells, and wherein the plurality of microfluidic channels permits an active fluid flow to the portion of the plurality of cell wells.

Disclosed herein is a method for providing a cell support system including forming a plurality of cell wells in a first block; forming a plurality of through holes through a second block; forming at least a portion of a plurality of microfluidic channels in at least one of the first block and the second block; coupling the first block and the second block such that the plurality of through holes are in fluid communication with at least one corresponding cell well of the plurality of cell wells, the plurality of microfluidic channels being configured to provide an active fluid flow with at least a portion of the plurality of cell wells.

Disclosed herein are embodiments of a cell support system, comprising a cell tray including a plurality of cell wells therein and a plurality of microfluidic channels in fluid communication with at least a portion of the plurality of cell wells, the plurality of microfluidic channels configured to provide a closed circuit for active fluid flow with the portion of the plurality of cell wells, and the cell wells configured to provide mechanically open access without interrupting the closed circuit for active fluid flow. In embodiments, the cell support system may further comprise a base assembly including a housing having an interior volume therein, the interior volume providing a controlled environment for the cell tray. The base assembly may include a hinged bezel for accessing the interior volume. In embodiments, the cell support system may further comprise a fluid delivery system in fluid communication with the microfluidic channels of the cell tray. In embodiments, the cell support system may further comprise at least one manifold interfacing between the fluid delivery system and the plurality of microfluidic channels.

### BRIEF DESCRIPTION OF SEVERAL VIEWS OF THE DRAWINGS

FIG. 1 shows a schematic cross-sectional diagram of the top and bottom components of a cell support system.

FIG. 2 shows a top view schematic of a cell support system.

FIG. 3 shows a perspective view of a cell support system.

FIG. 4 shows a top view of a cell support system and its microfluidics regions.

FIG. 5 shows a three-dimensional view of a cell support system interfacing with external fluid inflow and outflow systems.

FIG. 6 depicts an embodiment of an O-ring suitable for use with a cell support system.

FIG. 7 depicts an embodiment of a gasket suitable for use with a cell support system.

FIG. 8 depicts an embodiment of a chassis suitable for use with a cell support system.

FIG. 9 shows a perspective view of a cell support system.

FIG. 10 shows a perspective view of a cell support system.

FIG. 11 shows a perspective view of a base assembly for use with a cell support system.

FIG. 12 shows a perspective view of a fluid delivery system that may be used with a cell support system.

FIG. 13 shows schematically the path of fluid flow through a fluid delivery system and a cell support system.

FIG. 14 depicts one embodiment of a method for providing a cell support system.

### DETAILED DESCRIPTION OF THE INVENTION

A method and system for providing a cell support system are described. In one aspect, the method and system include providing a first block, a second block coupled to the first block, and a plurality of microfluidic channels. The first block includes a plurality of cell wells therein. The second block includes a plurality of through holes therethrough. In one aspect, the through holes may be between opposing faces of the second block. However, in another aspect, the through holes may not be between opposing faces. The plurality of holes are in fluid communication with at least one corresponding cell well of the plurality of cell wells. The plurality of microfluidic channels are in fluid combination with at least a portion of the plurality of cell wells and are configured to provide an active fluid flow with the portion of the plurality of cell wells. In another aspect, the method and system include investigating properties of a collection of cells. In this aspect, the method and system include introducing cells into a cell support system including a plurality of cell wells and a plurality of microfluidic channels therein. At least a portion of the plurality of cell wells is in fluid communication with the plurality of microfluidic channels. The plurality of microfluidic channels are configured to provide an active fluid flow with the portion of the plurality of cell wells. In this aspect, the method and system also include nourishing the cells in the cell wells with a fluid circulated actively along at least a portion of the plurality of microfluidic channels and performing at least one experiment on at least a portion of the collection of cells. In another aspect, the method and system include providing a cell support system. In this aspect, the method and system include forming a plurality of cell wells in a first block and a plurality of through holes through a second block. In this aspect, the method and system also include forming at least a portion of a plurality of microfluidic channels in at least one of the first block and the second block. The method and system also include coupling the first block and the second block such that the plurality of through holes is in fluid communication with at least one corresponding cell well of the plurality of cell wells. The plurality of microfluidic channels are configured to provide an active fluid flow with at least a portion of the plurality of cell wells.

The methods and systems herein are described in the context of particular cell support systems and methods for providing and using the cell support systems. One of ordinary skill in the art will readily recognize that the present invention is consistent with the use of systems having other and/or additional components not inconsistent with the descriptions herein. In addition, for clarity, the drawings herein are not to scale. The method and system are also described in the context of certain steps being performed in a certain order. For simplicity, steps may be omitted, combined, or described as having another order. However, one of ordinary skill in the art will readily recognize that the method and system are consistent with the use other and/or additional steps not inconsistent with the descriptions herein.

The systems and methods disclosed herein provide a pre-production platform for bioassays and biology imaging analysis. These systems and methods may permit the monitoring, for example, of large arrays of cell-based experiments. In embodiments, these systems and methods may allow researchers to perform time variation experiments on large pools of sample materials, with a reduction of time, space and cost as compared to more traditional methods for cell-based experiments.

Described herein are embodiments of systems and methods that provide an active microfluidic support for cell collections. Embodiments of the present invention include two-dimensional arrays of micron-sized wells containing cells and the nutrient solutions to support them. Such an arrangement enables automated processing as well as simultaneous monitoring and analyzing of a large matrix of cell collections. These arrays may be micromachined to be housed on a slide measuring 76.2×25.4 mm, the size of a standard microscope slide. A slide bearing an embodiment of the cell collection array is termed a “cell tray” herein. Stated differently, a cell support system may be included in and/or termed a cell tray. As used herein, the term “active flow” pertains to a cell support system where fluid is delivered to or extracted from some or all of the cell wells by use of an external positive or negative pressure source, for example a pressure pump, a pressurized tank, a vacuum pump, or the like. As used herein, the term “active support” relates to a cell support system where some or all of the cell wells receive active microfluidic flow. An active microfluidic system may be contrasted to a system using passive microfluidic flow, wherein fluid circulation is impelled by naturally occurring mechanisms such as gravity, capillary action, surface tension or the like to drive the flow.

Adapting the size of the array to the size of a standard microscope slide allows the arrays to be examined using a conventional microscope. Arrays configured for use with conventional microscopy may be designed with additional features to facilitate their handling. For example, a cell tray bearing an array may be configured with corners that are rounded or with indentations on the edges to allow easy pick-up or manual manipulation by an operator. Vertical tabs for the cell tray can be provided via the machining, molding, or by bonding that facilitate the use of automated grippers in slide handling robots. The cell tray may also be sized to rest on pedestals formed, for example, within a Petri-dish-like holder, permitting ready manipulation. While these systems and methods will be described by reference to an array sized to fit on a standard microscope slide, it is understood that other sizes and shapes of the array's housing may be produced to fit specific industry demands. While a small physical footprint is advantageous for certain purposes, it would be understood in the art that the housing may be formed in any size or shape to fit a particular piece of apparatus, or to provide a sufficiently large matrix for analytic purposes.

In embodiments, the cell support system disclosed herein may include a plurality of cell silos or wells into which may be loaded live cells. Wells are assembled into matrices so that thousands of wells may be provided, each containing a discrete cell population. Upwards of 7000 individual wells may be supported. In embodiments, cell wells may be of any suitable size or shape, for example, cylindrical or square or polygonal. Wells are advantageously scaled to micron sizes, for example, having volumes between 1 and 4 microliters, although it would be understood in the art that smaller wells could be provided in accordance with available methods for microfabrication if such wells could be serviced by ancillary devices such as micropipettes and sealing rings that were similarly miniaturized.

Cell wells, in embodiments of cell trays and other arrays as disclosed herein, are open on their top surface, so that the wells may be readily seeded with cells, for example under a laboratory hood. Following seeding with cells, the cell tray may be covered by a manifold assembly or by any appropriately sized and shaped cover so that the cells in the cell tray may be incubated. The aforesaid method permits live cells within the cell wells to become attached to their respective wells, so that they are not dislocated by subsequent maneuvers or by exposure to active perfusion or similar dynamic fluid environments. In embodiments, multiple cell trays may be seeded at one time in a laboratory hood or similar facility without a pump or a manifold, due to the open construction of the cell wells. It would be readily understood that seeding under a laboratory hood and/or remote incubation would be advantageous to free up the investigational microscope except for actual experiment time. It would be appreciated by artisans of ordinary skill, however, that cell tray seeding may take place with the tray placed on the microscope stage or in any convenient place. The cell tray may be pre-calibrated before seeding, to permit passive auto-focus, well-to-well navigation, microscopic scanning, and the like.

The open configuration of the cell wells in the cell tray also allows for experimental access to the cell wells before or after seeding. For example, cells may be extracted from the wells, or cells may be accessed with probes, micromanipulators, or other experimental tools, or cells may be treated with reagents in specific patterns, based on experimental protocols. For example, spotter technologies may be used to instill RNAi into different wells. In embodiments, the cell trays described herein may be pre-spotted with RNAi or the like to provide a substrate adapted for further experimentation. Other uses for the open cell well configuration will be apparent to those of ordinary skill in the art.

Wells may be arranged into discrete arrangements within regions on the cell tray. Each region may be configured with different well sizes or shapes, or different microfluidic properties. Such regional differences permit discrete, uniform sample populations to be created, so that multiple parallel isolated experiments may be conducted. In embodiments, fourteen regions of eighty (80) wells may be arrayed on a cell tray. In other embodiments, fewer regions of eighty (80) wells may be provided, for example, two or three regions. In other embodiments, regions may be arranged with any number of wells, for example with 8 wells, 12 wells, 100 wells or a multiple thereof. A region containing 8 wells may be particularly advantageous in applications where photo bleaching is a concern during, for example, fluorescence studies. It will be apparent to ordinarily skilled artisans that other arrangements of wells and regions may be provided, consistent with the needs of particular experimental protocols.

The wells on the cell tray are interconnected by a microfluidics network with channels that are tens of microns deep and require only nanoliter volumes of fluid. In embodiments, a cell channel may be 90 microns deep. In embodiments, the depth may range from 30 to 150 microns. Channel depth may be designed appropriately for the volume of fluid delivered to a collection of cell wells, recognizing that deeper channels may permit more efficient fluid flow, although fabricating deeper channels may be more difficult than fabricating shallow ones on a thin support matrix. In embodiments, a support matrix bearing microfluidic channels may have a thickness of approximately 200 microns; the relative depth of the microfluidic channels to the support matrix thickness may influence the structural integrity of the device.

In embodiments, an active microfluidic system may involve first delivering a volume of fluid to large “feeder” channels that lead to smaller fluidic pathways that interconnect each well. There are input “feeder” channels that allow fluid to flow into smaller channels and then diffuse into wells from one side. Output “scavenge” channels allow fluid to be removed from wells on the opposite side.

Disclosed herein is a cell support system including a cell tray and a manifold that encloses it. In embodiments, the cell support system is mechanically open and fluidically closed. Advantageously, this system may permit less reagent to be used, so that use of the present system may offer a low-cost alternative to current well-plate technologies. Advantageously also, this system may provide the basis for an improved “lab-on-a-chip” technology by allowing reagents to be automatically dispensed, and/or cells to be continually treated with reagents and cell culture media, features that current well-plate technologies may not permit. In embodiments, the cell tray of the cell support system may be placed within the manifold assembly. So positioned, the cell wells of the cell tray are accessible from the outside, so that the system is mechanically open. For example, cells may be loaded or seeded through micron-sized openings on the top of the support tray, and reagents may be added to the wells during tests. So positioned, the microfluidics of the cell tray are in fluid communication with an external fluid delivery system providing fluid inflow and outflow, with the path for fluid circulation being a closed one.

The manifold provides an interface between the microfluidics of the cell tray and an external fluid delivery system that may provide for fluid infusion and fluid withdrawal from the cell tray microfluidics. The cell tray may be configured with access ports that interconnect to the manifold, for example via O-ring connections. The manifold may be equipped with quick release fasteners to control preload on the O-rings to achieve a reliable, fluid-tight seal. Guide pins and the like may be provided on the manifold to align it properly with the cell tray. In embodiments, the manifold may be fabricated from biocompatible materials, for example, polycarbonates, polymethylpentene, and amorphous thermoplastic polyetherimide (e.g., Ultem®), and the like. In embodiments, the manifold may be autoclavable. In embodiments, the manifold may be opaque to minimize reflections and the like.

In an embodiment, the manifold may include a hinged cover glass, coated for example with indium tin oxide, to control access to the cell wells, to control temperature, to minimize contamination, and the like. So configured, the manifold provides a housing for the cell tray within which a controlled environment may be maintained. Temperature control may be facilitated by an integrated temperature feedback mechanism. An air port on the manifold may permit regulated gas flow into and out of the controlled environment. The manifold may contain infusion ports directed, for example to the various regions of cell wells on the cell tray, so that different fluids or reagents may be added to each region. Such infusion ports may be closed during normal operations, for example with a cover, a diaphragm or a one-way check valve. Consistent with the regional design of the cell tray, the manifold may be organized into regions as well. Regions within the manifold may be multiplexed, for example, to minimize tubing connections. Regions may also be isolated from each other to permit isolated experiments from region to region. Other features may be incorporated into the manifold to accommodate specific experimental needs, as would be apparent to persons having ordinary skill in the art.

Advantageously, the manifold may be constructed so that it interfaces with commercially available fluid delivery system components, such as the tubings and fittings used for high-pressure liquid chromatography and the like. In embodiments, the fluid delivery system may include a pump or a series of pumps to control fluid inflow and outflow. Pumps may be compactly made, so that they can fit conveniently on or under a laboratory bench. Pumps may, in embodiments, use commercially available syringe pumps for precision and reliability. In embodiments, miniaturized stand-alone pumps may be used that would be suitable for use in an incubator or under a laboratory hood. In yet other embodiments, micropumps may be incorporated into a portable, self-contained system appropriate for use in a non-laboratory setting, for example in the field for real-time testing or screening. Such portable systems may have utility in detecting bioweapons, environmental toxins, contaminants and the like.

The pumps may be controlled from a computer that is controlled from a user interface via, for example, a USB or other connection, in accordance with a fluid delivery program that regulates the fluid delivery system. The computer connections permit a plurality of fluid delivery systems to be controlled for a plurality of cell support systems. In embodiments, a fluid delivery program may include a series of preset routines for fluid delivery, including priming, feeding, infusing and purging the microfluidic channels of the cell tray. In embodiments, continuous and pulsed pumping modes may be available, including configurable pulse delays. A range of flow rates may be controllable, with pump rates ranging from 0.02 μl/s-1 ml/s continuous, for example. Vacuum offsets may be determined to compensate for environmental losses of fluid, via evaporation for example. Flow rates and volumes may be configured via the user interface. The fluid delivery system may further include infusion ports to allow reagents to be added to the inflow circuit, so that, for example, a set of parallel isolated experiments could be run using a single pump set. Infusion ports may be sealed from the rest of the fluid system with membranes or other sealants, or with static or dynamic valve systems, or by other mechanisms as would be understood by those of skill in the art. Other features may be incorporated into the fluid delivery system, for example a bubble trap to prevent bubbles from being introduced into the system, and cleaning/access ports to allow debris to be washed out but be sealed off from the rest of the system during operation.

In embodiments, the cell support system may be adapted for use with a microscope, for example a regular microscope, an inverted microscope, or any other optical device known in the art. A control system may allow the user to operate the microscope while managing the cell support system. In embodiments, controls could direct the well-to-well navigation of the microscope, its focus or focus offset adjustments, other camera adjustments, the filter wheel or shutter control, or other aspects of microscopy familiar to those of ordinary skill. The control system may also provide for experiment logging, configurable data indexing for import into an image analysis package, and the like.

In embodiments, disclosed herein is a microfluidic cell support system, comprising a first support substrate containing a plurality of cell wells, a second support substrate affixed to the first support substrate with a plurality of access holes therethrough, each access hole being in fluid communication with a cell well on the first support substrate, and an active microfluidic circulatory path in fluid communication with at least one of the cell wells, whereby the active microcirculatory path provides a flow of fluid to the cell well. In embodiments, the cell tray of the cell support system may be machined to incorporate a resistance network to control the flow of fluid evenly across all the regions of the cell tray and across all the wells in each region. As an example, a resistance network, may comprise channels entering the cell wells that are greater than one order of magnitude smaller in diameter than the upstream channels. In embodiments, the cross-section of the well inlet channels may be two orders of magnitude smaller than the cross-section of the upstream channels. The order of magnitude difference in geometry ensures that the pressure drop through the upstream channel is negligible as compared to the pressure drop across the inlet of each well. A bubble bypass may be included that prevents bubbles from obstructing the microfluidic channels on the cell tray, and to prevent bubbles from interfering with the parallel resistors controlling even fluid flow. In embodiments, there may be flushing channels to allow fresh fluid to flush the system. In embodiments, the microfluidic channels abutting the cell wells may be configured to prevent cells from washing out of the cell wells on the outflow side of the fluid path. For example, a zig-zag or other departure from a straight line may be designed so that the fluid flow does not dislodge the cells from cell wells.

In embodiments, disclosed herein is a method of investigating properties of a collection of cells, comprising introducing cells into a plurality of cell wells formed in a microfluidic cell support system, nourishing the cells in the cell wells with fluid circulated along a microfluidic circulatory path, and performing an experiment on the collection of cells.

In embodiments, a cell support system may include a support substrate onto which a plurality of cell wells may be formed. The cell wells may be in fluid communication with an active fluidic system that is also formed on the support substrate. While certain of the fabrication techniques described herein may be familiar to artisans of ordinary skill in micromachining, the disclosure that follows sets forth specific techniques for the production of a microfluidic cell support system representative of inventive systems and methods. Following this general description, more specific features of a cell support system may be understood with reference to the Figures.

In general, the cell support substrate may be formed from two materials that are bonded together after being etched using different masks. Substrate materials may include fused silica, soda-lime glass, silicon, germanium, sapphire, polymethylmethacrylate (PMMA), other carbon-based or silicon-based polymers, and the like. Flexible substrates that are easily molded such as Sylgard 184 Poly(dimethylsiloxane) (PDMS) and the like may also be used. Substrate materials may be selected depending on particular physical properties, including the desired optical transmission properties for electromagnetic radiation at a particular wavelength. Substrate materials may also be chosen based upon desirable chemical or fluidic control properties such as hydrophobicty, hydrophilicity, or the propensity for cells to adhere to a certain type of substrate.

As an example, an optically transparent material (e.g., an optically flat borosilicate glass) in block form (wafer) may be used for the bottom portion of the support substrate. The next steps pertain to photolithography as one method of fabrication. A layer of photoresist is applied to the surface of the wafer. A mask with a desired pattern is placed over the layer of photoresist. The photoresist is then exposed to ultra-violet light (or other appropriate source) transferring the image from the mask to the wafer surface. The bottom substrate (glass in this case) may then be etched, creating the desired pattern on the bottom block. The process is repeated using a second mask (Mask 2), which after etching forms the microfluidic channels. (The photomask is created by a photographic process and developed onto a glass substrate. The lowest cost masks use ordinary photographic emulsion on soda lime glass, while Chrome on quartz glass is used for the high-resolution deep UV lithography). This masking layer may be a metal, a photoresist silicon oxide, or any other substance suitable for photolithography. Using, for example, anisotropic plasma etch, channels with a depth of 20 microns are etched into the bottom block. In embodiments, channel depth may range, for example from 10-30 microns. In certain embodiments, a lithographic mask may be computer-designed and directly transferred to a photoresist using, for example, a laser scanning microscope. In other embodiments, a two-axis Ronchi ruling or the like may be used to expose a cross-grated pattern on the photoresist layer. Alternatively, a lithographic shadow mask may be substituted for the Ronchi grating. A shadow mask may, for example, consist of a two-dimensional array of square or circular apertures. In other embodiments, a holographic exposure process may also be used to generate a crossed-grating interference pattern in the photoresist. Variations of these and other techniques will be familiar to artisans of ordinary skill.

In certain embodiments, the photoresist may be exposed using a narrow-wavelength light, a laser light or a broadband white light. Shadowed regions on the photoresist that were not exposed to the light may thereupon remain as surface structures in the photoresist after the developing process. A negative photoresist would work in an opposite way. Using this process, a two-dimensional ordered array of square, circular or other geometric shaped regions may be obtained.

It is understood that other fabrication processes may be employed instead of a negative or a positive photoresist. A positive photoresist can be substituted along with a negative of the aperture mask. In addition, the cell wells may be fabricated using other techniques, including e-beam or deep UV lithography in PMMA substrate or any other optical substrate material. Other techniques may be used to fabricate features of the cell support system as appropriate, including hot press, embossing, injection molding or stamping on suitable glass or plastic substrates.

In embodiments, the masks are unique designs that result in groups or regions being formed on the bottom block. For example, a pattern may be formed with 14 regions formed onto the bottom block, each region containing wells having wells with micron-dimensioned sizes, such as 50 micron, 100 micron, 200 micron or 300 micron target sized. wells. It would be understood by those of ordinary skill in the art that wells of any suitable size may be fabricated, consistent with the needs of a particular experimental modality. In embodiments, well sizes within a range from 50 to 300 microns may be useful for the experimental purposes for which a cell support system is designed. Etched microfluidic channels may interconnect wells grouped in the same region. In this example, 14 different regions exist, each with its own microfluidic circulation. Hence, 14 different experiments may be run simultaneously.

A top block may be bonded as a cover for the bottom block. The top block may be made from silicon or from other materials familiar in the art. For example, a double-sided polished silicon wafer of approximately 200 microns thickness may be patterned with shallow channels on one side, using photolithography (Mask 3). The orientation of these channels matches the channels on the bottom block, as described above. The masking layer may be photoresist silicon oxide, a metal or any other suitable substance. In one embodiment, the shallow channels on the cover block may be etched 4 microns deep with a DRIE etcher (a high-aspect ratio plasma etch). The cover block may be patterned again on the same side using photolithography and a different mask (Mask 4). The pattern of Mask 4 provides for deep channels. The masking layer may be photoresist silicon, a metal or any other suitable material.

The cover block may include through-holes that are aligned with the cell wells on the bottom block. The through-holes may be patterned by etching the side of the cover block that has not yet been etched. A mask (Mask 5) may be designed for the through-holes so that they are properly aligned with the cell wells. Etching, for example a DRIE plasma etch, is carried through the entire silicon wafer. In embodiments, the silicon top block may be opaque within the visible wavelength spectrum but capable of transmitting infrared light. In this embodiment, infrared may be used to visualize the cell wells in the bottom block through the silicon so that the top may be properly aligned with the bottom. When both blocks have been suitably etched, they may be bonded together to form the completed support substrate. For example, the silicon cover block may be aligned to better than 1 micron using a wafer alignment tool and anodically bonded to the patterned borosilicate glass slides. In embodiments, tighter tolerances may be achieved for alignment, or less precise alignment may be elected. In embodiments, the top silicon layer may be fabricated to have smaller length and/or width dimensions so that the glass block underneath it is visible, highlighting the two-layer construction of the device.

Other options for forming the channels may be appreciated by artisans of ordinary skill in the art. For example, polymers such as SU-8 may be used to build up wells and channels on a glass base. In embodiments, such polymers may be deposited via lithographic methods permitting great precision. A cover block may be thermally bonded to the base bearing the deposited polymers. In another embodiment, channels and wells may be formed in a glass base plate using other methods, such as ultrasonic drilling, laser machining, shot/sand blasting, wet etching, and the like. Such methods may be equally applicable to forming the through-holes in the cover block, particularly if it were made of glass.

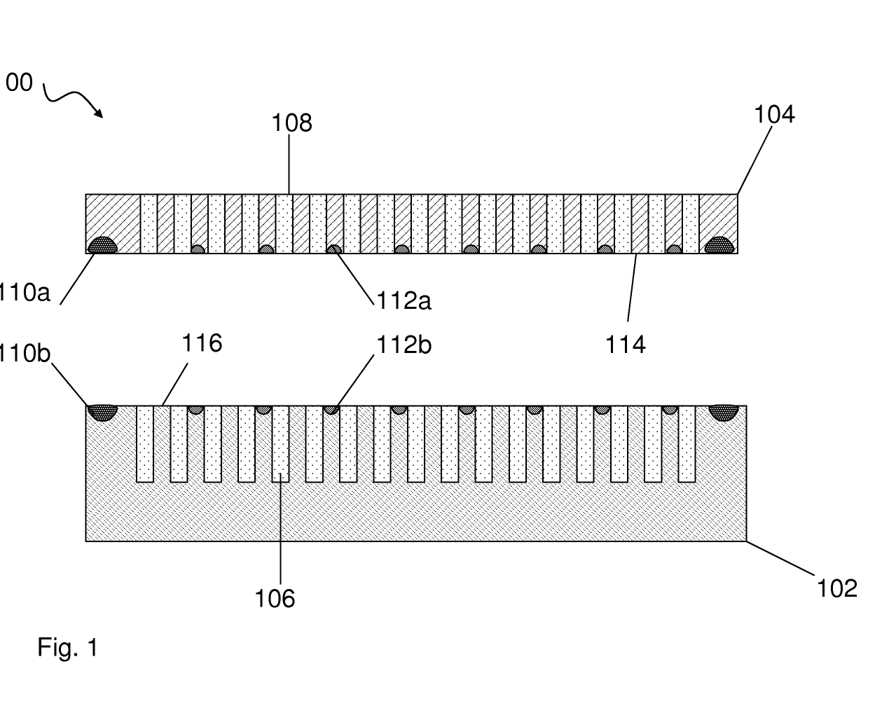

With reference to FIG. 1, certain features of a cell support system 100 may be appreciated. FIG. 1 depicts schematically an embodiment of a cell support system having a lower block 102 of support substrate and an upper block 104 of support substrate. A plurality of cell wells 106 have been formed in the lower block 102, using techniques such as those described above. A plurality of through-and-through access holes 108 have been fabricated in the upper block 104, using techniques such as those described above. When the upper block 104 and the lower block 102 are properly aligned, the access holes 108 are in continuity with the cell wells 106. Providing microfluidic circulation to the cell wells is a network of microfluidic channels. This network comprises a set of primary channels and secondary channels, as illustrated in this figure. There may be further branchings of the circulatory network, comprising tertiary, quaternary channels, and so on.

FIG. 1 shows, in an embodiment, how the primary and secondary channels may be formed in the upper block 104 and lower block 102, so that when the two blocks are properly aligned, a complete primary or secondary channel arises. In the upper block is a plurality of upper half-shapes 110a of primary channels. These correspond to a plurality of lower half-shapes 110b of primary channels, so that when the two blocks are properly aligned, a complete primary channel is formed. Similarly, in the upper block is a plurality of upper half-shapes 112a of secondary channels. These correspond to a plurality of lower half-shapes 112b of secondary channels, so that when the two blocks are properly aligned, a complete secondary channel is formed. Alternatively, the channels may be entirely formed in the lower block 102, with no corresponding formations in the upper block 104. The upper block has a lower surface 116 and the lower block has an upper surface 114 that are joined together to form the completed support substrate for the cell support system. In the depicted embodiment, the primary channels give rise to the smaller secondary channels. Secondary channels may in turn give rise to smaller-yet tertiary channels and so on.

It is understood that the arrangement of microfluidic channels may be designed for a specific use. While channels at two-dimensional or three-dimensional right angles may be conveniently constructed, channels and subchannels may be arranged in any shape and may take off from each other at any angulation. For example, it may be desirable to form channels and subchannels as a network having acute two-dimensional and three-dimensional angles. It may be desirable to have channels and subchannels arranged in a pattern similar to the vasculature and microvasculature of the human body. In addition, channels need not be formed only on the horizontal plane. Other channel arrangements may also be suitable, for example with primary channels in the vertical plane or heading diagonally. Other arrangements of cell wells may also be desirable. The illustrative embodiments described above show cylindrical cell wells positioned at right angles to the joining surfaces of the upper and lower substrate blocks. The cell wells need not be cylindrical, nor need they be positioned at any particular angle. Instead, their shape and orientation may be determined by the needs of a particular experimental situation.

For example, a cell well may be bifurcated, so that the population of cells placed therein divides itself into two discrete cell collections. Bifurcation may be carried out using a number of techniques familiar to artisans of ordinary skill. For example, multiple substrates may be spotted into a well, each permitting attachment of a specified type of cell. Or for example, the interior surface of a well may be modified to permit attachment of discrete cell collections. As another example, the well geometry may be modified to promote the attachment of separate cell colonies, by use of a partition or the like. A bifurcated cell well may additionally be fed by two different microfluidic circulatory systems, one providing nutrition for example, and the other providing exposure of one half of the bifurcated well to a particular reagent. In this way, each well could contain both an experimental collection of cells and a control. It is understood that the same techniques that may permit bifurcation of a cell well may also permit the cell well to be divided into multiple segments (i.e., trifurcated, or multifurcated).

Other design choices for the arrangement of microfluidic systems and cell wells may be readily apparent to artisans of ordinary skill in the art adapting the systems and methods disclosed herein to particular situations.

Moreover, while embodiments of a cell support system have been described that use only two support substrate blocks, more complex cell support systems may be designed to meet particular needs. For example, three or more support substrate blocks may be formed with cell wells and access holes in continuity. A microfluidics system may be three-dimensionally designed, running for example through the three or more layers of support substrate blocks to provide circulation to the cell wells. Such a system may, for example, provide inflow of nutrients through an upper microfluidics circulation, and outflow of spent medium through a lower microfluidics circulation.

In the depicted embodiment, a simple design is proposed with a construction that has a minimal number of channels, with cells in proximity to the air surface. As would be understood by artisans of ordinary skill in the art, designs with greater complexity may be utilized, depending on the goals for the system and on the fluid modeling programs underlying the particular design.

In other embodiments, dual or multiple microfluidics networks may be fabricated that permit the exposure of cells in the cell wells to a plurality of substances, including, for example, nutrient media and test reagents. In an embodiment, cells may be exposed to substances that enter the wells through the microfluidic inlet channels, including such materials as cell culture media or reagents, as would be appreciated by artisans of ordinary skill in the art. In an embodiment, substances maybe introduced into the wells from the top, as the wells are open to air through the access holes 108. In an embodiment, effluent substances draining from the cell wells may be collected for further experimental purposes, for furthering cell growth, and the like. For example, the effluent from the cell wells may be analyzed for the presence of a particular substance like a reagent, a protein, an antibody, a small molecule, etc. In this way, the response of the cells to certain experimental conditions may be ascertained. As another example, a product of cellular metabolism may be collected from the cell well effluent as an indication of cell physiology or pathophysiology, or as a desirable byproduct to be accumulated for further processing. As yet another example, the effluent from the cell wells may be removed to allow the cells optimal contact with nutrients, growth factors and other desirable substances in the medium, and to minimize their exposure to spent medium containing undesirable wastes. As would be understood in the art, more complicated inflow and outflow systems may be designed to permit exposure to a variety of reagents, to allow reagents to mix before entering a well, and the like. Flow channels may incorporate valves and switches to facilitate more complex designs.

In one embodiment, the lower block of support substrate 102 may be fabricated from borosilicate glass, anodically bonded to the upper block of support substrate 104 made from silicon. The two blocks may be joined together by other means, too, such as an adhesive bonding. If adhesive bonding were used, other materials may be employed for the upper and lower substrate blocks 102 and 104. In embodiments, the upper block of support substrate 104 may be a photoetched piece of metal, such as Kovar or Invar that matches the thermal coefficient of expansion of the Quartz or Glass CellTray base. In embodiments, adhesive could be screen printed on to the top piece to assure it is put in the correct areas. When a metal such as Kovar is used, which is susceptible to corrosion problems when used with aqueous solutions such as Basal Medium, the upper substrate block 104 may be chrome and/or nickel plated following etching. In other embodiments, the upper substrate block 104 may be another piece of quartz or glass, similar to or identical to the material used for the lower substrate block 102.

The upper substrate block 104 may be bonded to the lower substrate block 102 via a number of means, as will be understood by artisans of ordinary skill in the art. For example, optical contacting, adhesive, or high temperature bonding may be used. In embodiments, the upper substrate block 104 and the lower substrate block 102 may be joined by thermal compression bonding, or by roller lamination, where heat is the primary bonding agent. In embodiments, adhesives may be placed on one or both layers, which are then aligned and pressed together.

Options exist that may facilitate the mass production of certain embodiments of the present system. In embodiments, the lower block 102 may be fabricated from a polymer. In these embodiments, compression molding or embossing may be used, for example with a heated platen press using a nickel electroform made from a silicon master. In such embodiments, the lower block 102 may be cut free from the molded blank using equipment familiar in the art, e.g., a dicing saw or CNC Mill. In other embodiments, the lower block may be fabricated via injection molding, using for example a mold that also has a nickel electroform insert for the micron order size features. In such embodiments, minimal additional work may be needed to produce the finished lower block 102, for example, trimming off sprues from mold injection ports and other finishing techniques familiar in the art. In embodiments that are fabricated using injection molding, materials such as PMMA, Polycarbonate, Cyclic Polyolefin such as Zeonor or Topas, and the like, may be used. It may also be molded from one or two part silicone polymers such as PDMS at low pressure using a silicone master, a nickel electroform from a silicone master, or from a physically micromachined metal mold using machining methods such as carbide mills, electrodischarge machinings, laser machining, or diamond point machining. Such a flexible substrate might be bonded or adhered to a standard glass or quartz slide as a rigid, transparent substrate.

In embodiments, the upper block 104 may be fabricated using a variety of techniques. For example, it could be compression molded in a similar manner to the lower block 102. With compression molding, the multiplicity of through-holes 108 may not be readily fabricated; in certain embodiments having several thousand through-holes 108, such fabrication may not be feasible using an injection mold. Using methods similar to those disclosed above, the tip could also be fabricated from PDMS or similar flexible, low modulus polymers that are biologically compatible. Hence, other fabrication techniques using mechanical means may be employed. In embodiments, the upper block 104 may be fabricated with through-holes 108 formed, for example, with compression or injection molding, extending partially through the upper block 104. In such embodiments, the upper block 104 may then be machined or polished along the uncut surface so that the through-holes 108 are exposed. Alternatively, a laser could be used to cut through the uncut surface of the upper block 104, thereby exposing the through-holes 108 while avoiding physical contact. In other embodiments, the entire through-hole 108 (200 μm thick) may be cut through the upper block 104 after the upper block 104 has been laminated to the lower block 102. Using this technique, the through-holes 108 would be optimally aligned with the cell wells 106. It is understood, however, that the laser employed may desirably be carefully tuned to avoid damaging the lower block 104. Instead of using a laser, one could punch the through-holes 108 using a mechanical punch.

In embodiments where polymer is selected as the substrate for molding the lower block 102 and/or the upper block 104, it may be desirable to coat the internal surfaces of the wells and/or channels with additional polymer layers: for example the wells 106 and channels 112, 114 may have a hydrophilic coating that forms the top of the cell wells 106. Artisans of ordinary skill in the art will appreciate that a number of polymer coating methodologies are available to perform the polymer coating in a single layer or as multiple layers. Other coatings, including aqueous based coating, plasma or other means of changing surface energy could also be used to modify the behavior of the polymers used in the upper or lower blocks 102, 104, or for the cell wells 106.

While the depicted embodiment shows primary channel grooves 110a and 110b in the upper and lower blocks, and secondary channel grooves 112a and 112b in the upper and lower blocks, it would be understood that primary and secondary channels could be fabricated entirely within the upper or the lower block, or could be shaped, for example, in the lower block and then sealed over by the lower surface 114 of the upper block or vice versa. Other arrangements for designing and constructing the channels would be apparent to artisans of ordinary skill in the art.

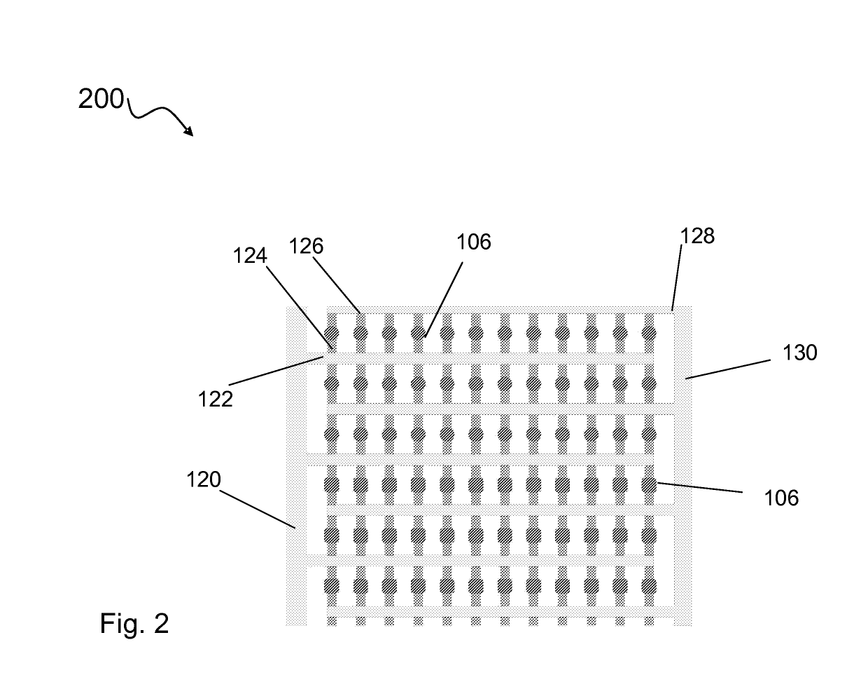

With reference to FIG. 2, a schematic of the fluid drainage system for a cell support system 200 is depicted. Large input “feeder” channels 120 for fluid flow carry fluid from the reservoir towards the plurality of cell wells 106 of the fluid drainage system is depicted. Note that cell wells may be cylindrical, rectangular, or any other shape, as would be understood by those of ordinary skill in the art. Main inflow channels 120 connect to the reservoir (not shown) containing the fluid to be conveyed to the cell wells 106. The inflow fluid (containing nutrient media or the like) passes from the main inflow channels 120 to the primary inlet channels 122 (which correspond to the primary channels 110 shown in FIG. 1). The inflow fluid passes from the primary inlet channels 122 into the secondary inlet channels 124 (which correspond to the secondary channels 112 shown in FIG. 1). In embodiments, the cross-sectional area of a main inflow channel 120 is at least one order of magnitude greater than the cross-sectional area of a primary inlet channel 122. In embodiments, the cross-sectional area of a primary inlet channel 122 is at least one order of magnitude greater than the cross-sectional area of a secondary inlet channel 124. In another embodiment, the cross-sectional area of a primary inlet channel 122 is approximately two orders of magnitude greater than the cross-sectional area of a secondary inlet channel 124. This relationship among the sizes of the inflow channels may comprise a resistance network to equalize the flow entering a selection of cell wells.

As depicted in FIG. 2, fluid enters the cell wells 106 from the secondary inlet channels 124. Secondary outlet channels 126 (corresponding to the secondary channels 112 shown in FIG. 1) carry waste fluid away from the cell wells 106. The secondary outlet channels 126 drain into the primary outlet channels 128 (also corresponding to the primary channels 110 shown in FIG. 1), which in turn convey the waste fluid into main outflow channels 130. In embodiments, the main outflow channels 130 may be subjected to negative pressure through their connection to the drainage system to enhance the efflux of waste fluid.

In embodiments, a pressure drop may be maintained across the main inflow channels 120, the primary inlet channels 122 and the secondary inlet channels 124. The pressure drop in each channel system may be designed to ensure a balanced delivery of fluid to all wells 106 in the matrix, and/or to meter the flow rate into each well 106 so that each receives the same nominal inlet flow. In certain embodiments, the pressure drop in the main inflow channels 120 and/or the primary inlet channels 122 may be designed to be much less than the pressure drop of the secondary inlet channels 124. An arrangement of pressure drops may be similarly engineered for the outflow side of the system, as would be understood by artisans of ordinary skill in the art. It is understood that arrangements to maintain inflow-side or outflow-side pressure drops depend on multiple variables, including flow rates, geometry, configuration and the like. As an example, a pressure drop of in the range of 1×10³ to 1×10⁴ dyne/cm², may be maintained in certain embodiments.

In embodiments, the secondary inlet channels 124 and the secondary outlet channels 126 may be shallow relative to the dimensions of the cell wells 106, to ensure that any cells residing in the wells 106 are retained in the wells 106 with the circulation of fluid in the microfluidic system, and to prevent the cells from being displaced from the wells 106. For channels having a depth and a width, a depth of the inlet channels 124 and outlet channels 126 may be combined with a preselected channel width for any given well size or well matrix, so that an appropriate pressure drop and flow rate are obtained. If the channels are rounded rather than rectangular in shape, a depth axis and a width axis may be selected for the inlet channels 124 and the outlet channels 126 to produce the appropriate pressure drop and flow rate. In embodiments, the channels have a shallow depth when compared to the width. Dimensions may be varied to produce differences in pressure drop and flow rate, as would be understood by artisans of ordinary skill in the art. As an example, the depth of the channels may be configured so that a cell is unable to escape from the cell well. For example, the depth of the channel may be made less than the diameter of the cell. In one embodiment, for example a 20 micron diameter cell would not be able to navigate a 5 micron channel.

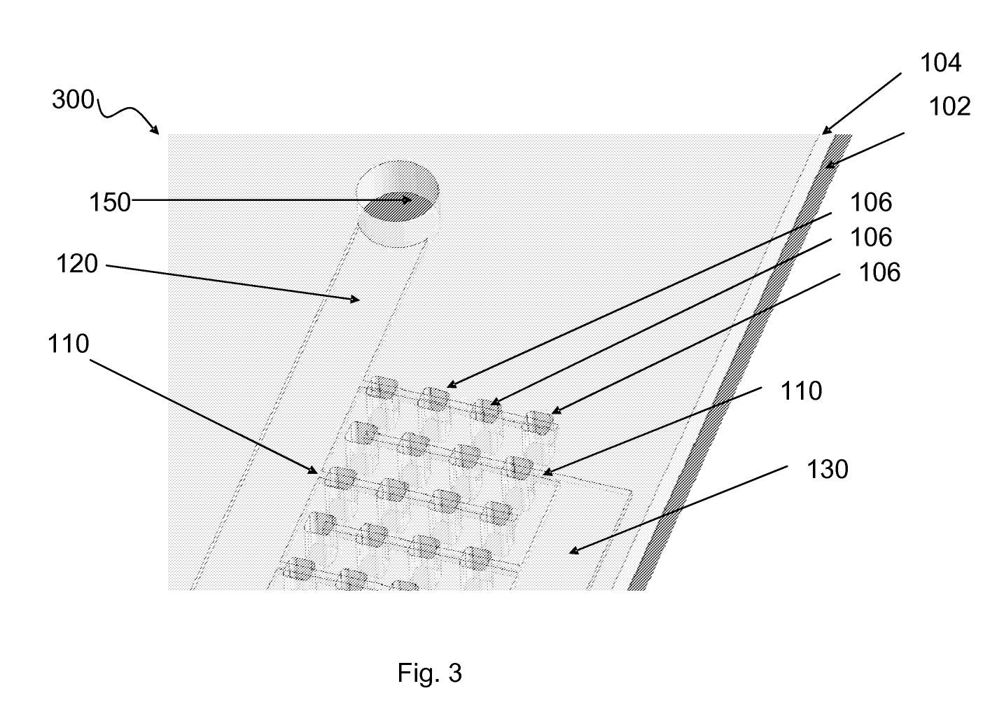

With reference to FIG. 3, the three dimensional arrangement of an embodiment of a cell support system 300 may be appreciated. FIG. 3 shows the deployment of a plurality of cell wells 106 in the lower block 102 covered by the upper block 104. A fluid connection 150 is in fluid communication with the fluid reservoir (not shown), to allow the inflow of externally-provided fluids such as nutrient media into the system. The fluid passes into a main inflow channel 120 and thence into primary channels 110 and secondary channels (not shown). On the outflow side, waste fluid passes into secondary channels (not shown) to primary channels 110, and thence into a main outflow channel 130 for removal from the system. On the outflow side, a fluid connection (not shown) is in fluid communication with the outflow passages that provide external fluid removal.

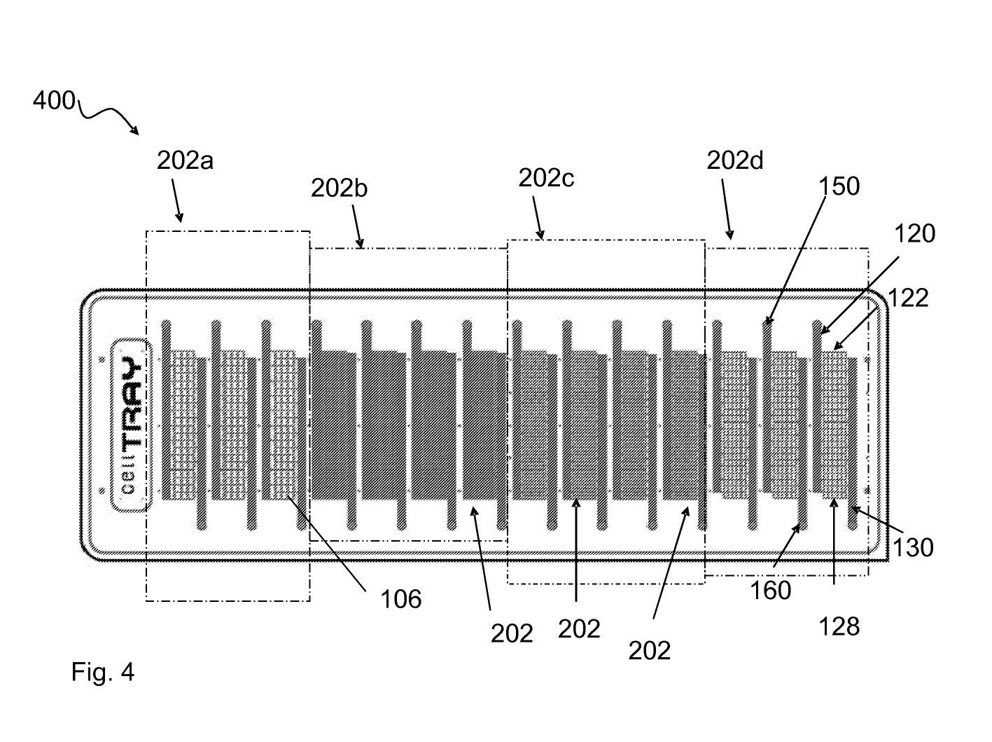

FIG. 4 depicts a schematic overview of an embodiment of a cell support system 400. In the depicted embodiment, there are 14 regions 202 (only selected regions 202 being identified on the Figure), each containing an array of cell wells 106, and each with its own inflow and outflow tracts that connect to inflow and outflow conduits. As described previously, the inflow tract may contain a main inflow channel 120 bringing fluid in through a reservoir connection 150, which has connections to an external source of inflow fluid, e.g., nutrient media. As described above, fluid enters each inflow tract through the main inflow channel 120, then entering the primary inlet channel 122, then the secondary inlet channel (not shown), thereupon entering the cell wells 106. The outflow tract contains a system of outflow channels to drain waste fluid, e.g., spent media containing waste products or desirable cellular metabolic products, from the cell wells 106. As described above, fluid enters each outflow tract by passing from the cell wells 106 into the secondary outlet channels (not shown) into the primary outlet channels 128 which connect to the main outflow channels 130. The main outflow channels are in fluid communication with the drainage system through a drainage connection 160, thereby allowing the waste fluid to be removed from the cell support system.

In the depicted embodiment, each of the fourteen regions 202 contains a plurality of cell wells 106. In embodiments, all wells 106 within a particular region 202 have the same dimensions. Well dimensions may be varied for different purposes, as would be appreciated by artisans of ordinary skill in the art. In the depicted embodiment, several groups of regions 202a, 202b, 202c, 202d are shown. Each of these groups of regions shown here receives a different fluid input. In embodiments, differences in fluid input may involve different nutrient media, different components or additives to the media, different growth factors, different pharmacological agents, different microbes, or the like. Each group of regions contains a set of cells residing in a set of cell wells 106. The wells may have similar sizes in each region or group of regions, or the wells may be sized differently from region to region or from group to group. There may be different populations of cells in the different regions or groups of regions, or different numbers of the same types of cells, or the like. In embodiments, the outflow from each region or group of regions may have a different composition, reflecting the differences in input solutions, the differences in cell populations, the differences in cell densities, the difference in experimental conditions, and the like.

It is understood that while the depicted embodiment shows 14 regions 202, any convenient number of regions 202 may comprise the cell support system 400. The regions 202 may be grouped into groups to allow varying a certain number of experimental parameters, or the regions 202 may each be varied individually. Moreover, while the embodiment of a cell support system 400 in FIG. 4 is shaped as a rectangle, any shape may be selected consistent with the needs of a particular set of experiments. For example, a round cell support system 400 may be designed that may bear a number of regions 202 with a central inflow channel and with peripheral outflow channels, or vice versa. Or a square cell support system 400 may bear a number of regions 202 with inflow and outflow systems arranged to take advantage of the support system geometry. Other examples will be readily apparent to artisans of ordinary skill in the art.

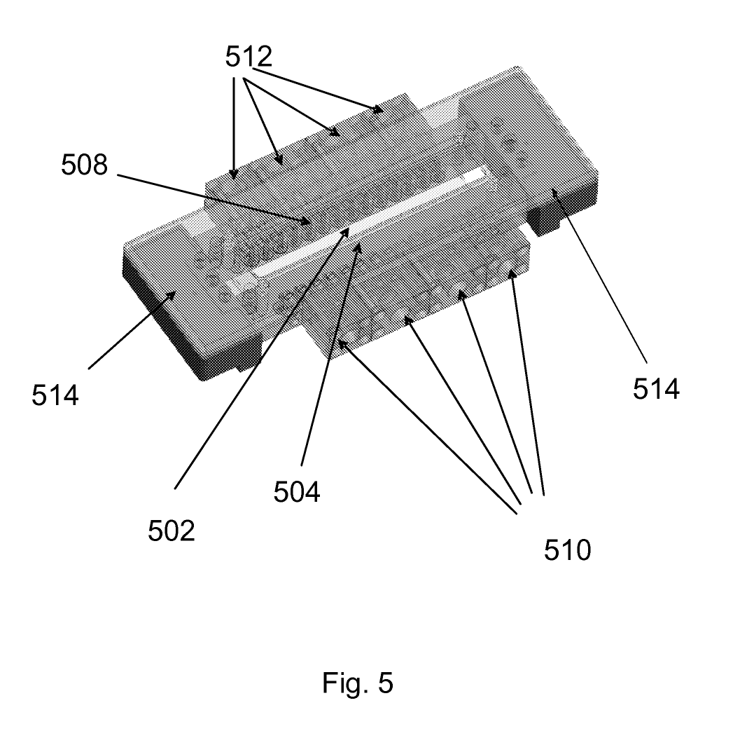

FIG. 5 depicts an embodiment of a cell support system 502 (shown as a ghost shape in this figure) interfacing with an inflow manifold 504 in fluid communication with the main inflow channels (not shown) that were depicted in previous figures. With reference to FIG. 5, the inflow manifold 504 may be in fluid communication with a set of input ports 510, here four input ports 510, although any number may be designed for a particular embodiment. On the outflow side, an outflow manifold 508 is in fluid communication with the main outflow channels (not shown) that were depicted in previous figures. With further reference to FIG. 5, the outflow manifold 508 may be in fluid communication with a set of output ports 512, here four output ports 512, although any number may be designed for a particular embodiment. In embodiments, a set of O-rings, gaskets or the like, may be positioned in between the inflow channel (not shown) of the cell support system and the inflow manifold 504, or between the outflow channel (not shown) of the cell support system and the outflow manifold 508 to ensure a tight seal.

In embodiments, the input ports 510 may be in fluid communication with one or several fluid reservoirs or other containers for agents to be provided to the cells in the cell support system 502 on the inflow side. In embodiments, the output ports 512 may be in fluid communication with one or several conduits for waste materials, spent media, and the like, optionally allowing the collection of samples that are removed from the wells of the cell support system 502. In embodiments, the fluid path on the inflow side may have its flow or pressure regulated by external controls that interface with the inflow system (e.g., pumps, vacuums, and the like). In embodiments, the fluid path on the outflow side may have its flow or pressure regulated by external controls that interface with the outflow system (e.g., pumps, vacuums, and the like). It would be understood by artisans of ordinary skill in the art that there may be further controls of inflow and outflow paths, including feedback circuits between the two, all to be determined by the particular uses for which the cell support system is employed. In the depicted embodiment, the cell support system 502 may be supported by or encased in a support housing 514. As will be shown below, the support housing 514 may be adapted for inclusion in a chassis (not shown) which may bear external reservoirs for inflow materials, external containers for outflow materials, controls for inflow and outflow circuits, and the like.

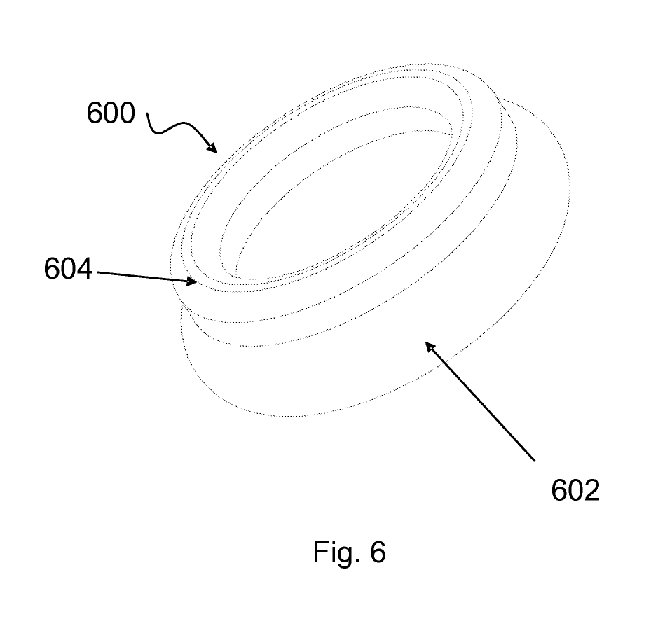

FIG. 6 depicts an embodiment of an O-ring 600 that may be used to ensure a tight seal between a main inflow channel and the inflow manifold, or a main outflow channel and the outflow manifold. In the depicted embodiment, the O-ring 600 may be retained within a dovetailed o-ring groove to ensure it stays in the manifold and does not stick to the cell support system. An O-ring 600 may be made, for example, with 40 durometer rubber (comprising such materials as fluoroelastomers such as Viton® and the like, silicone, butadiene and acrylonitrile copolymers such as BUNA-N, and PolyChloroTriFluoroEthylene (e.g., Kel-F®)). In the depicted embodiment, the base 602 is standard o-ring, but an added lip 604 section is molded in for sealing and compliancy, as shown in FIG. 6. In embodiments, a two-shot co-molding process may be used, where the base 602 O-ring is a firmer rubber and the exposed sealing portion 604 is softer. Advantageously, a sealing ring 600 of this design may allow the use of a manifold with less strict machining tolerances.

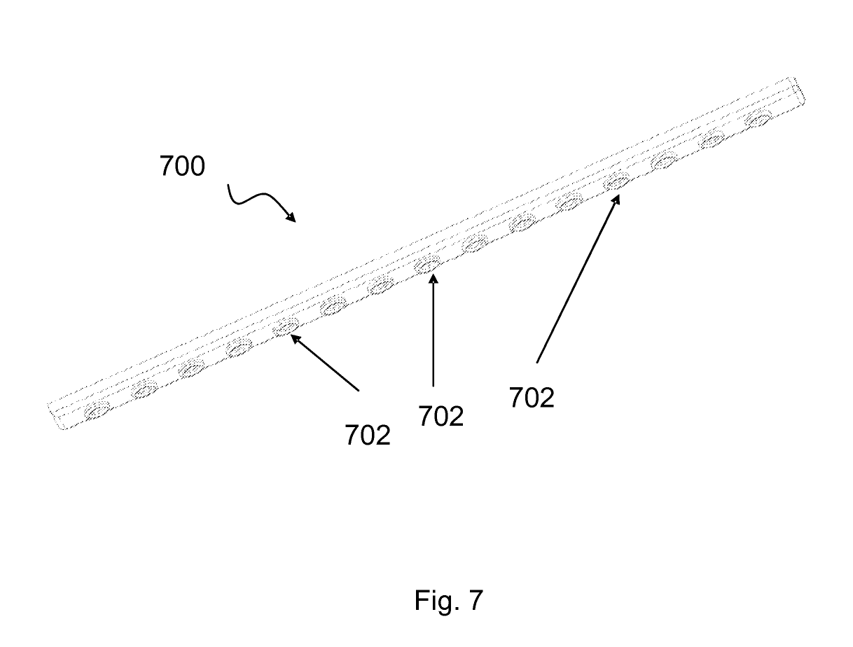

FIG. 7 depicts an alternate arrangement for a sealing system that may be positioned between the inflow or outflow manifold and the main inflow or outflow channels, respectively. In the depicted embodiment, a gasket 700 contains a plurality of sealing rings 702. The gasket 700 may be made in one piece from, for example, custom-molded silicone. In embodiments, the gasket may seal as a flat gasket to the base of the manifold (not shown), with a raised compliant set of sealing rings 702 that each interface with an inflow or outflow channel (not shown).

Advantageously, machining for this design may be simple, as maintaining one height across the entire base via one cutting operation may be less difficult than multiple o-ring grooves. As would be understood by artisans of ordinary skill in the art, a gasket 700 may be fabricated using a simple mold. In embodiments, the gasket 700 may be made with a dovetail molded into it that matches the manifold to ensure that it is retained in the manifold and does not stick to the cell support system. In embodiments, after de-aired silicone is poured in the mold, it may be topped with a flat glass plate or the like, clamped to assure flatness, and cured in an oven. The gasket 700 may be molded from silicone or from other appropriate polymers. The durometer of the rubber to be used for the gasket 700 may be selected based on factors including compliance, sealing and combinations thereof. In another embodiment, a two part mold may be used that has raised compliant sections on both sides of the gasket 700. In this case, the manifold base may be machined to have corresponding o-ring type gaskets around each feed hole. The described gasket 700 could also be injection molded using well known techniques. In embodiments, the gasket 700 may have a metal or rigid polymer frame co-molded with it to ensure stiffness and retention in the gasket recess.

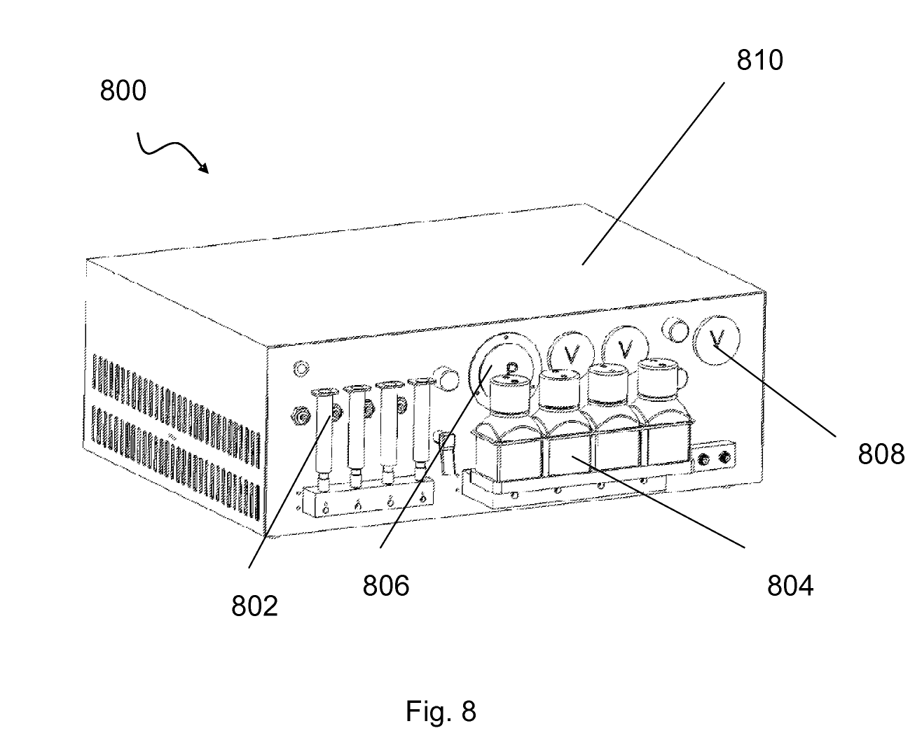

FIG. 8 shows an embodiment of a chassis 800 adapted for containing a cell support system and the related inflow systems, outflow systems and control systems. In the depicted embodiment, the chassis 800 comprises a housing unit 810 that contains the cell support system (not shown) and associated fluid paths and circuitry, some of which have been described previously in more detail. In the depicted embodiment, the housing unit 810 bears on its external aspect certain external fluid containers for fluid input and fluid output. In more detail, a series of external fluid reservoirs 802 may be seen, which may provide fluid input for the cell support system within the housing unit 810. The pressure or the flow rates for input fluid may be controlled by various controllers. As depicted here, the input fluid may have its pressure controlled, with input pressure readings available on a pressure gauge 806. On the outflow side, a set of waste collection bottles 804 may be borne on the housing, allowing, for example, the external collection of output fluid. Fluid flow on the output side may be subjected to positive or negative pressure, for example, with a pressure reading available on a vacuum gauge 808. As will be understood by artisans of ordinary skill in the art, a wide variety of chassis 800 arrangements may be contemplated that would interface advantageously with the cell support system described herein.

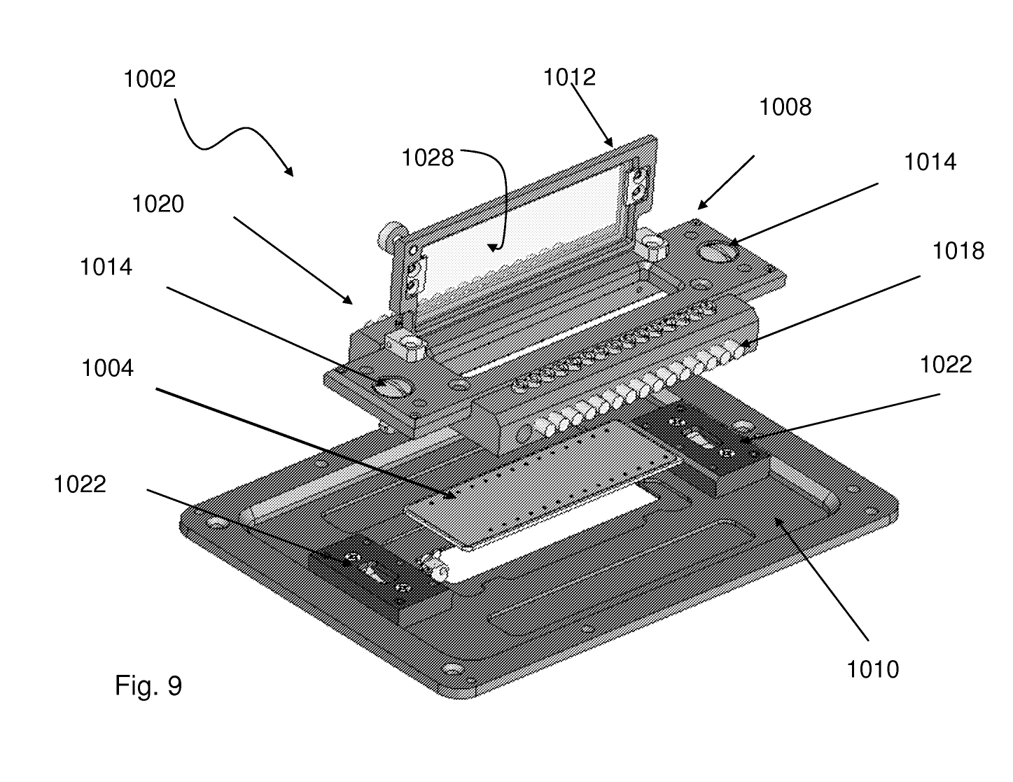

FIG. 9 shows an embodiment of a cell support system 1002 comprising a base assembly 1010, a manifold assembly 1008, and a cell tray 1004. The base assembly 1010 bears two attachment bases 1022 between which the cell tray 1004 may be positioned. The attachment bases 1022 permit attachment of the manifold assembly 1008 using, for example, a set of fasteners 1014. The fasteners 1014 may be configured as quarter turn fasteners, for example to ensure repeatable preload. In embodiments, a hinged bezel 1012 may be provided, permitting access to the cell tray 1004 for cell loading. In the depicted embodiment, the hinged bezel 1012 is fitted with a central glass viewing area 1028. In the depicted embodiment, a series of fluid intake ports 1018 are provided in the manifold assembly 1008. In the depicted embodiment, a series of fluid outflow ports 1020 are also provided. The fluid intake ports 1018 and the fluid outflow ports 1020 permit attachment to an external fluid delivery system (not shown). In the depicted embodiment, the cell support system 1002 may have a low profile that is suitable for use with a standard microscope. In particular, the base assembly 1010 may have a low profile that accommodates either a cell tray 1004 or a standard microscope slide (not shown).

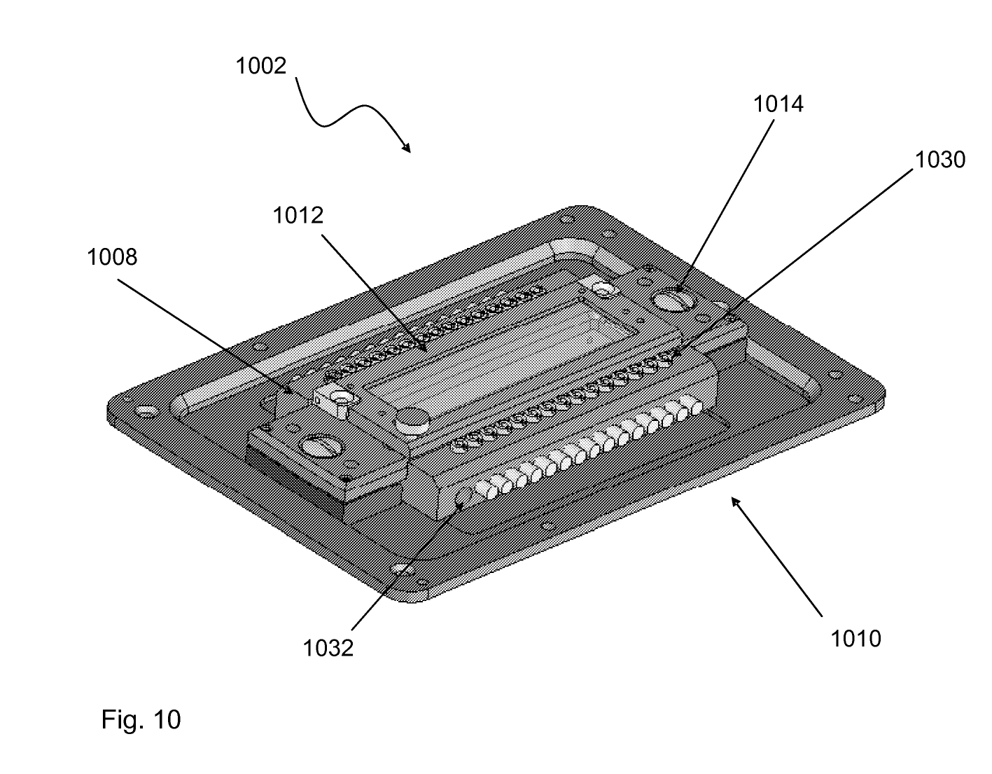

FIG. 10 shows in more detail an embodiment of a cell support system 1002 with the manifold assembly 1008 attached to the base assembly 1010 to enclose an interior volume having a controlled environment. A cell tray 1004 (not shown) may be positioned within the interior volume. In the depicted embodiment, a series of ports 1030 are provided for example for adding reagents to the fluid line heading into the cell tray (not shown) and its microfluidics. In the depicted embodiment, an air inlet port 1032 is provided, into which temperature-controlled air, carbon dioxide, or other gas mixes may be delivered. In the depicted embodiment, the hinged bezel 1012 is securely fastened, for example by a fastening screw 1014.

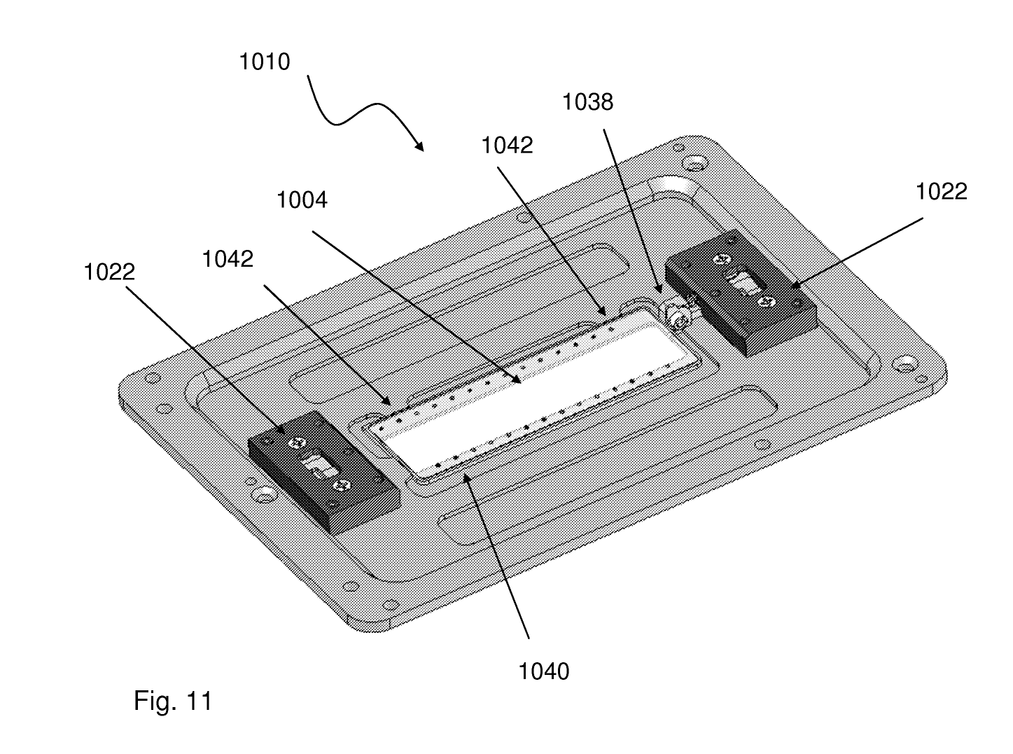

FIG. 11 shows in more detail an embodiment of a base assembly 1010 for use with a cell support system as described in previous figures. As depicted in this Figure, a cell tray 1004 is positioned between two attachment bases 1022. One of the attachment bases 1022 supports a spring-loaded wedge with ejector 1038 to which the cell tray 1004 may be affixed. In embodiments, the spring-loaded wedge with ejector 1038 may permit easy attachment and quick release of the cell tray 1004 from the rest of the base assembly 1010. In embodiments, an arrangement of support tabs 1042 and cutouts 1040 may be provided that permit manual access to the cell tray 1004 and that permit a more stable support. In the depicted embodiment, the cell tray 1004 is supported along all its edges.

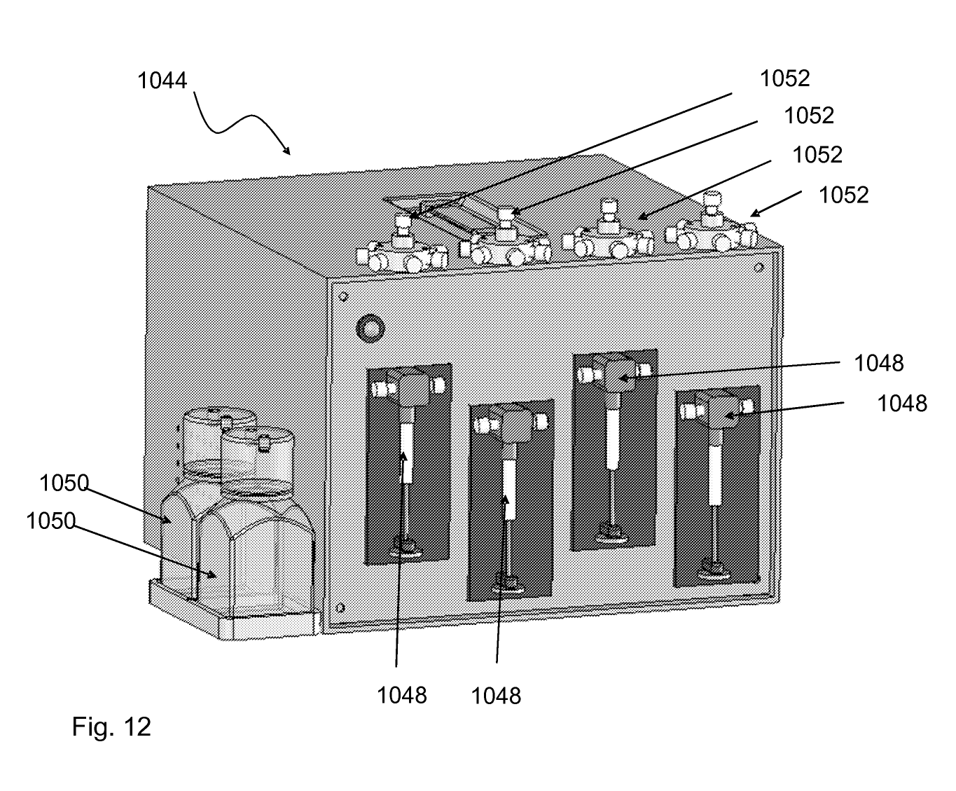

FIG. 12 depicts an embodiment of a fluid delivery system 1044 that may be used with the cell support system (not shown) depicted in previous figures. In a depicted embodiment, a series of syringe pumps 1048 insert and remove fluid from the cell support system. The syringe pumps 1048 are selected to provide volumes of fluid that are appropriate for the microfluidics of the cell support system, for example 250 μl volume. Specifications for syringe pumps 1048 may pertain to the size of the syringe and its total volume, the timing of a full stroke, and a/or the resolution. In embodiments, a syringe pump may allow both and infuse and a withdrawal mode. Stir in sizes may vary from, for example, 50 μl to 50 ml. A full stroke may take place in a time period ranging from 0.5 seconds to 16 minutes. Resolution may include 30,000 steps per full stroke. Particular specifications may be established for particular experimental purposes, as would be understood in the art. Reservoirs 1050 are provided for fresh media and for waste media. The depicted embodiment shows a set of seven-way manifolds 1052 to direct the inflow toward the cell support system (not shown), and to collect the spent media removed from the cell support system. As would be understood by artisans of ordinary skill, other arrangements for fluid delivery systems 1044 may be readily envisioned, consistent with the cell support systems disclosed herein. A fluid delivery system 1044 may be of any suitable size. The depicted embodiment, for example, may have dimensions of 11 inches in depth, 11 inches in width, and 8 inches in height, or any other suitable dimensions.

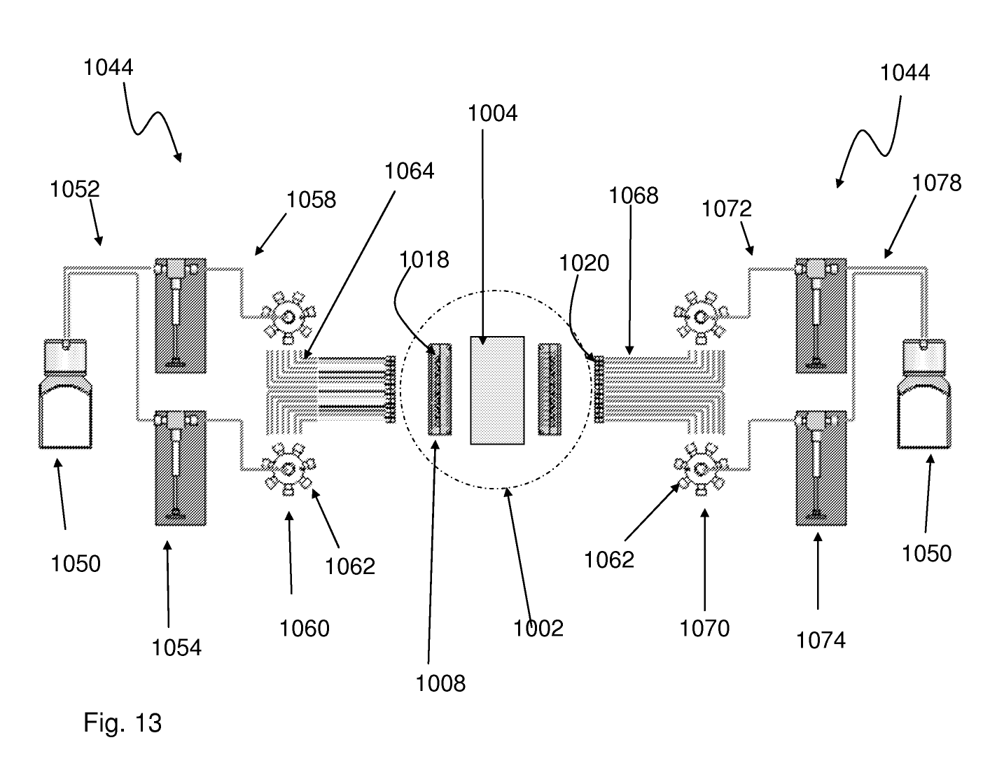

FIG. 13 shows schematically a diagram of fluid flow through a system comprising a fluid delivery system 1044 and a cell support system 1002. In this diagram, the inflow side is to the left and the outflow side is to the right. A reservoir 1050 is depicted at the left side of the diagram to contain infusate. The infusate flows through inflow tubing 1052 into a set of dispenser pumps 1054. The infusate then flows through a set of infusion pump tubes 1058 to enter the infusion manifold 1060. The infusion manifold as depicted in this figure has seven manifold heads 1062 each one of which feeds a separate inflow channel 1064. In the depicted embodiment, there is a total of 14 inflow channels 1064. Each inflow channel 1064 interfaces with a fluid intake port 1018 on the manifold assembly 1008 of the cell support system 1002. Each fluid intake port 1018 interfaces with an input feeder channel (not shown) on the cell tray 1004, as described in more detail herein. After flowing through the microfluidic network of the cell tray 1004, as described in more detail herein, the spent media is collected in the outflow scavenge channels (not shown) of the cell tray 1004. In the outflow scavenge channels of the cell tray interface with the fluid outflow ports 1020 on the manifold assembly 1008. Each fluid outflow port 1020 communicates with an outflow channel 1068 which in turn feeds a manifold head 1062 on the outflow manifold 1070. The outflow manifold 1070 is in communication with the outflow pump tubing 1072 from which the spent media is withdrawn by the outflow pump 1074. The waste tubing 1078 conveys the spent media into the reservoir 1050 from which it may be collected. Additional features may be incorporated into the fluid flow paths on the inflow or the outflow side. For example, there may be a check valve or other entry point on the inflow side to allow the input of reagents into one or more of the inflow channels. In embodiments, such check valves may be incorporated into the manifold assembly. As another example, withdrawal ports or catches may be provided on the outflow side from which samples may be withdrawn from one or more of the outflow channels. The addition of reagents or other experimental components may be controlled manually or in an automated way, for example by having electronic valves that are programmed to allow a timed delivery or a timed withdrawal. In other embodiments, reagents may be added to or withdrawn from the cell tray itself while fluid perfusion is ongoing. This is possible because the cell support system 1002 in embodiments is mechanically open while being fluidically closed.

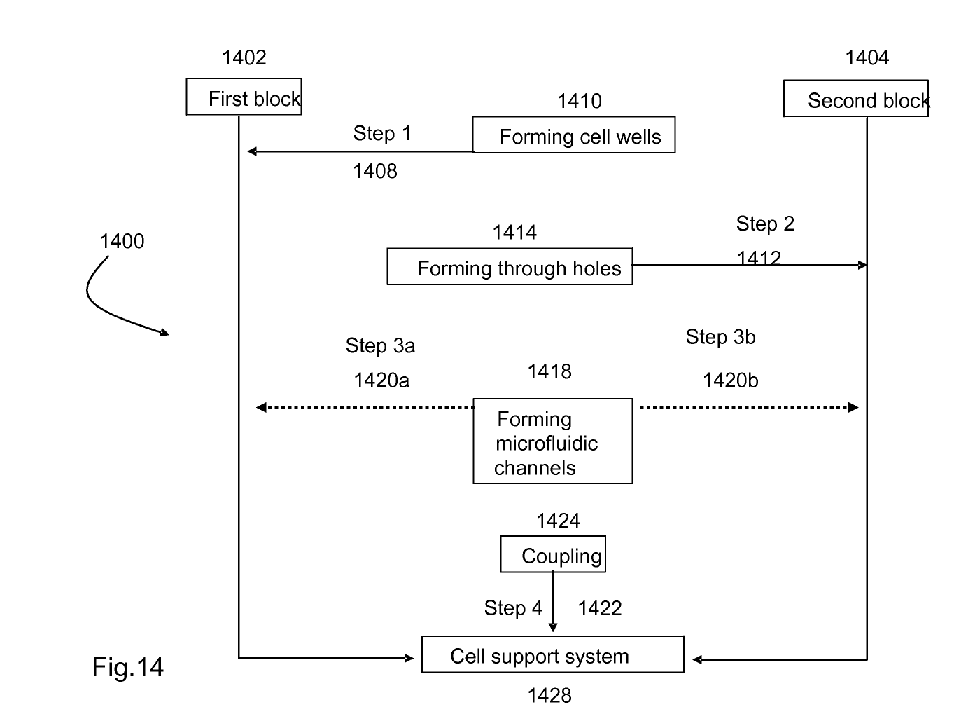

In embodiments, a cell tray 1004 may be fabricated in a number of ways, as would be understood by those of ordinary skill in the art. FIG. 14 provides a flow chart 1400 depicting schematically one embodiment of a method for fabricating a cell support system. As depicted in this figure, a first block 1402 and a second block 1404 may be provided. As a first step 1408 a process 1410 of forming cell wells may be performed to create cell wells in the first block 1402. As a second step 1412, a process 1414 of forming through holes in the second block 1404 may be performed. As a third step, a process 1418 of forming microfluidic channels may be performed. The process 1418 of forming a plurality of microfluidic channels may be carried out in at least one of the first block 1402 (Step 3a, 1420a), or in the second block 1404 (Step 3b, 1420b). The process 1418 may be carried out so that certain microfluidic channels are formed in both the first block 1402 and the second block 1404, thereby combining elements of step 3a (1420a) and step 3b (1420b). In embodiments, the plurality of microfluidic channels formed by the process 1418 may be configured to provide an active fluid flow with at least a portion of the plurality of cell wells formed by the process 1410 of step 1 (1408). As a fourth step 1422, a process 1424 may be carried out whereby the first block 1402 and the second block 1404 are coupled to form a cell support system 1428. In embodiments, the process of coupling 1424 may be performed so that at least one of the plurality of through holes in the second block 1404 formed by the process 1414 of step 2 (1412) is in fluid communication with at least one corresponding cell well of the first block 1402 formed by the process 1410 of step 1 (1408).

Again with reference to FIG. 13, embodiments of a cell tray 1004 may comprise a lower glass wafer and an upper silicon wafer. The glass wafer and the silicon wafer are processed as follows:

As a first step, a glass wafer may be provided for processing as the lower component of the cell tray 1004. In embodiments, borosilicate float glass 1.1 mm thick and 100 mm in diameter may be used. As a second step, a nickel vanadium layer 80 nm thick may be deposited on the glass wafer. This layer will be used for patterned fiducials and branding. As a third step, a layer of photoresist may be deposited. The photoresist may be exposed with a pattern for nickel etch. As a fourth step, the nickel may be etched. As a fifth step, the photoresist may be removed and the wafers may be cleaned. As a sixth step, a chrome layer may be deposited. The chrome is intended to protect the glass and patterned nickel. As a seventh step, a layer of photoresist may be deposited. The photoresist may be exposed to a pattern for glass etch. As an eighth step, the chrome may be etched. As a ninth step, glass may be etched. As a tenth step, the chrome and resist may be stripped and the wafers may be cleaned.

A silicon wafer may be used for the upper component of the cell tray 1004. As a first step, the silicon wafer may be processed. In a second step, approximately 1 μm of silicon may be oxidized. The oxidized layer will serve as a protective layer for a shallow silicon etch. As a third step, a layer of photoresist may be deposited. The photoresist may be exposed to a pattern for a shallow silicon etch. As a fourth step, the oxide layer may be etched. As a fifth step, the resist may be stripped and the wafers may be cleaned. As a sixth step, a layer of chrome may be deposited. Chrome will serve as a protective layer for a deep silicon etch. As a seventh step, a layer of photoresist may be deposited. The photoresist may be exposed to a pattern for a deep silicon etch. As an eighth step, the chrome layer may be etched. As a ninth step, the resist may be stripped and the wafers cleaned. In the tenth step, a layer of photoresist may be deposited. In the eleventh step, the silicon may be etched with a through pattern to about 140 nm. In a twelfth step, the resist may be stripped to expose the masking chrome layer. In a thirteenth step, the silicon may be etched with a deep channel pattern to about 86 μm. Through holes are now etched complete. In a fourteenth step, the masking chrome layer may be removed to expose the oxide pattern. In a fifteenth step, the oxidized silicon may be etched with a shallow channel pattern to about 4 μm. Finally, the wafers are cleaned and a surface is oxidized.

Following the fabrication of the upper layer and the lower layer of the cell tray 1004, the two layers are aligned and bonded. As would be understood by those of ordinary skill in the art, other substrates may be used for the upper and lower layers. As would be understood by those of ordinary skill in the art, other fabrication techniques may also be used.

While the invention has been described in connection with certain preferred embodiments, other embodiments would be understood by one of ordinary skill in the art and are encompassed herein.

1.-25. (canceled)

26. A cell support system comprising:
a first block including a plurality of cell wells therein, the cell wells each being dimensionally adapted for containing a discrete cell population of live cells;
a second block coupled with the first block, the second block including a plurality of through holes therethrough, the plurality of through holes being in fluid communication with at least one corresponding cell well of the plurality of cell wells; and
a plurality of microfluidic channels in fluid communication with at least a portion of the plurality of cell wells, the plurality of microfluidic channels configured to provide an active fluid flow with the portion of the plurality of cell wells.

27. The cell support system of claim 26, wherein each of the cell wells is between 30-50 microns deep.

28. The cell support system of claim 26, wherein each of the cell wells contains a fluid volume between 1 and 4 microliters.

29. The cell support system of claim 26 further comprising:
at least one manifold coupled with the plurality of microfluidic channels.

30. The cell support system of claim 29 wherein the at least one manifold provides fluid communication with at least one external reservoir.

31. The cell support system of claim 29 further comprising:
at least one access port providing access to the at least one manifold without interrupting its fluid communication with the plurality of microfluidic channels.

32. The cell support system of claim 29 wherein the at least one manifold isolates a first portion of the plurality of cell wells from a second portion of the plurality of cell wells.

33. The cell support system of claim 26 wherein at least a portion of the plurality of microfluidic channels are formed within the first block.

34. The cell support system of claim 26 wherein the plurality of microfluidic channels include a plurality of inlet channels characterized by an inlet size and at least one main channel characterized by a main channel size, the at least one main channel size being at least an order of magnitude larger than the inlet channel size.

35. The cell support system of claim 26 wherein the plurality of microfluidic channels include at least one inlet, the plurality of microfluidic channels being configured to support a pressure differential between the at least one inlet and the portion of the plurality of cell wells.

36. The cell support system of claim 26, wherein the plurality of microfluidic channels are configured to be microfluidically closed and the plurality of cell wells are configured to be mechanically open to experimental access before or after seeding the plurality of cell wells with the discrete population of live cells.

37. A cell support system, comprising:
a cell tray including a plurality of cell wells therein and a plurality of microfluidic channels in fluid communication with at least a portion of the plurality of cell wells, the plurality of microfluidic channels configured to provide a closed circuit for active fluid flow with the portion of the plurality of cell wells, and the cell wells configured to provide mechanically open access without interrupting the closed circuit for active fluid flow.

38. The cell support system of claim 37, further comprising a base assembly including a housing having an interior volume therein, the interior volume providing a controlled environment for the cell tray.

39. The cell support system of claim 37, wherein each of the cell wells has a volume between 1 and 4 microliters.

40. A method for providing support to a cell population of live cells, comprising:
providing a cell tray including a plurality of cell wells therein and a plurality of microfluidic channels in fluid communication with at least a portion of the plurality of cell wells, the plurality of microfluidic channels configured to provide a closed circuit for active fluid flow with the portion of the plurality of cell wells, and the cell wells configured to provide mechanically open access without interrupting the closed circuit for active fluid flow;
introducing a cell population of live cells into at least a portion of the cell wells in the cell tray; and
directing nutrient media fluid through the microfluidic channels to the cell wells of the cell tray and permitting spent media outflow from the cell wells of the cell tray in active fluid flow, the active fluid flow providing support to the cell population of live cells.
